(19) United States
(12) Patent Application Publication
(10) Pub. No.: US 2026/0339285 A1
(43) Pub. Date: Oct. 29, 2026

(54) SYSTEM AND METHOD FOR PROCESSING DENTAL PROSTHESIS USING WEB SERVER

(71) Applicant: Huvitz Co., Ltd., Anyang-si (KR)

(72) Inventors: Seung Suk HA, Anyang-si (KR); Young Gil SHIM, Anyang-si (KR); Weon Joon LEE, Anyang-si (KR)

(21) Appl. No.: 19/439,406

(22) Filed: Jan. 4, 2026

(30) Foreign Application Priority Data
Jan. 8, 2025 (KR) 10-2025-0002677

(51) Int. Cl. G05B 19/18

(52) U.S. Cl. CPC G05B 19/182; G05B 2219/45145

(57) ABSTRACT

Disclosed are a system and a method for effectively driving a prosthesis processing device using a web server. The system includes: a user terminal (60) through which commands for performing functions of the dental prosthesis processing system are input; a web server (50) located at a remote location, connected to the user terminal (60) and one or more milling devices (40a to 40f) through a communication network (70), and performing a tool path data generation operation to produce a processing path of a processing tool according to status information including device characteristics and usage status characteristics of the processing tool mounted on milling devices (40a to 40f); and one or more milling devices (40a to 40f) that transmit their own usage status characteristics to the web server (50) and process a workpiece into the shape of a prosthesis according to the tool path data generated by the web server (50).

### CROSS-REFERENCE TO RELATED APPLICATION

This application claims priority from Korean Patent Application No. 10-2025-0002677 filed on Jan. 8, 2025, which is incorporated herein by reference in its entirety.

### TECHNICAL FIELD

The present invention relates to a system and a method for processing dental prostheses using a web server, and more particularly, to a system and a method for effectively driving a prosthesis processing device using a web server.

### RELATED ART

Prosthesis treatment is a dental treatment that restores tooth function by removing damaged portions of teeth and mounting dental prostheses, i.e., artificial teeth, in the areas where the damaged portions have been removed.

To fabricate dental prostheses, a three-dimensional scanner is used to obtain the three-dimensional shape of the oral structure where the prosthesis will be placed, and the shape of the required prosthesis is designed using CAD (Computer Aided Design). Next, based on the obtained prosthesis shape data, a milling device operated by numerical control (NC) and CAM (Computer Aided Manufacture) software processes a workpiece (also referred to as a “block”) such as a ceramic block into the prosthesis shape (see Korean Patent No. 10-1854730, Korean Patent Publication No. 10-2010-0003646, etc.).

FIG. 1 shows the configuration of a conventional dental prosthesis processing system. As shown in FIG. 1, a conventional dental prosthesis processing system includes a computation and control unit 30 equipped with CAM software, two or more milling devices 40a to 40f, an input unit 10, a display unit 20, and so on.

The computation and control unit 30 is equipped with CAM software and controls the milling devices 40a to 40f according to user commands input through the input unit 10 to fabricate prostheses. Specifically, the CAM software performs (1) a nesting operation to place the shape of the prosthesis to be processed on the workpiece, i.e., the block, and (2) a tool path generation operation to calculate the processing path of the processing tool based on the structure (diameter, size, etc.) of the processing tool mounted on the milling devices 40a to 40f. The milling devices 40a to 40f are controlled according to the nesting and tool path data. The computation and control unit 30 may be a conventional computer or laptop equipped with the CAM software.

The two or more milling devices 40a to 40f process workpieces such as a ceramic block into the prosthesis according to commands from the computation and control unit 30. Since the milling devices 40a to 40f are primarily used by dentists or dental hygienists in hospitals rather than experts who professionally operate the processing devices, they are also referred to as “Chair Side milling devices.” Therefore, the milling devices 40a to 40f should be relatively easy to use by non-experts, and it is desirable to enable automatic placement of the prosthesis shape on the block or automatic prosthesis processing.

The input unit 10 is a device through which commands for operating the dental prosthesis processing system and/or three-dimensional shape data of the oral structure are input. The display unit 20 is a device that displays the prostheses being processed in the dental prosthesis processing system and the status of the milling devices 40a to 40f, and so on.

As shown in FIG. 1, in a conventional dental prosthesis processing system, up to six milling devices 40a to 40f are connected to the computation and control unit 30, i.e., a computer equipped with CAM software, for control and monitoring.

Meanwhile, in recent years, according to improvements in CAM software and an artificial intelligence (AI), methods of mounting CAM software on a remote web server and using the CAM software through a communication network such as the Internet have been studied. That is, a method is being researched in which a patient's oral scan data is transmitted to a web server equipped with CAM software through a communication network, the web server performs CAD data editing for designing the prosthesis, nesting operations, and tool path generation operations, and the obtained data is then transmitted back to the milling device through the communication network to process the prosthesis.

In such a method using a web server, if information on the block and milling device model to be used is provided, CAD data, nesting data, tool path data, etc., are automatically generated accordingly. However, in the actual milling device where the prosthesis is processed, the tool path should be determined based on the information of the actually mounted block or the actually mounted tool, rather than the block or the milling device's model information. That is, in a conventional dental prosthesis processing system, the computation and control unit 30 equipped with CAM software directly controls the milling devices 40a to 40f to fabricate prostheses, and at this time, nesting data and tool path data are generated using only general model information of the block and milling devices 40a to 40f. As a result, the precision of the generated data is not satisfactory, and when processing the block to fabricate the prosthesis, the dimensional precision of the fabricated prosthesis is not satisfactory.

### PRIOR ART DOCUMENTS

(Patent Document 1) Korean Patent No. 10-1854730
(Patent Document 2) Korean Patent Publication No. 10-2010-0003646

### SUMMARY

An object of the present invention is to provide a dental prosthesis processing system and a method for effectively driving a prosthesis processing device using a web server.

Another object of the present invention is to provide a dental prosthesis processing system and a method capable of obtaining more accurate nesting data and tool path data using a web server and processing prostheses more accurately using the data.

To achieve the above objects, the present invention provides a dental prosthesis processing system comprising: a user terminal through which commands for performing functions of the dental prosthesis processing system are input; a web server located at a remote location, connected to the user terminal and one or more milling devices through a communication network, and performing a tool path data generation operation to produce a processing path of a processing tool according to status information including device characteristics and usage status characteristics of the processing tool mounted on milling devices; and one or more milling devices that transmit their own usage status characteristics to the web server and process a workpiece into the shape of a prosthesis according to the tool path data generated by the web server.

Preferably, the dental prosthesis processing system further comprises a display unit that displays the prosthesis and the status of the milling devices.

Preferably, the milling devices are controlled by the web server.

Preferably, the device characteristics of the processing tool are characteristics of the processing tool itself including one or more of an initial or reference position, diameter, length, and movement trajectory of the processing tool, and the usage status characteristics of the processing tool are characteristics of the processing tool that occur during the use of the processing tool mounted on the milling devices.

The present invention also provides a dental prosthesis processing method comprising the steps of: nesting step of selecting a block for processing a prosthesis and placing a shape of the prosthesis designed in a desired shape on the block; transmitting status information including usage status characteristics of one or more milling devices to a web server through a communication network; generating tool path data for producing a processing path of a processing tool at the web server according to the status information including device characteristics and the usage status characteristics of the processing tool mounted on the milling devices; and controlling the milling devices according to the generated tool path data to process the block into a shape of the prosthesis.

Preferably, the usage status characteristics of the processing tool mounted on the milling devices are transmitted from each of the milling devices to the web server through the communication network.

According to the dental prosthesis processing system and the method of the present invention, more accurate nesting data and tool path data can be obtained using a web server, and prostheses can be processed more accurately using the data.

### BRIEF DESCRIPTION OF THE DRAWINGS

FIG. 1 is a diagram showing the configuration of a conventional dental prosthesis processing system.

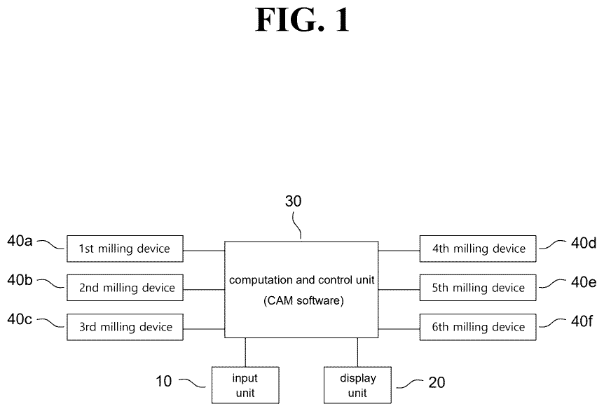

FIG. 2 is a diagram showing the configuration of a dental prosthesis processing system using a web server according to an embodiment of the present invention.

FIG. 3 is a diagram showing a state in which the shape of a prosthesis is placed on a block according to an embodiment of the present invention.

FIG. 4 is a diagram for explaining a dental prosthesis processing method using a web server according to an embodiment of the present invention.

### DETAILED DESCRIPTION

Hereinafter, the present invention will be described in detail with reference to the accompanying drawings. In the accompanying drawings, the same reference numerals are assigned to components that perform the same or similar functions as in the prior art.

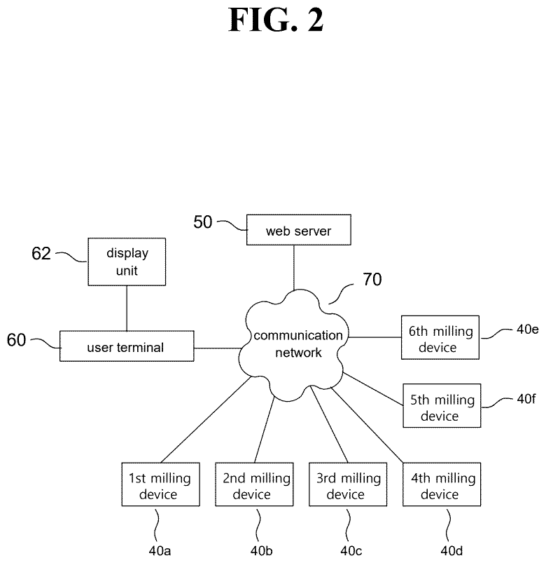

FIG. 2 is a diagram showing the configuration of a dental prosthesis processing system using a web server according to an embodiment of the present invention. As shown in FIG. 2, the dental prosthesis processing system according to the present invention includes a user terminal 60, a web server 50, and one or more milling devices 40a to 40f, and may further include a display unit 62 as necessary.

The user terminal 60 is a device through which commands for performing functions of the dental prosthesis processing system are input, and conventional input devices such as a keyboard, a touch panel, a mouse, etc., may be used for inputting commands. Additionally, as necessary, at the user terminal 60, (1) designing (editing) the shape of a prosthesis in a shape desired by the user, and/or (2) a nesting operation to place the shape of the prosthesis to be processed on a workpiece, i.e., a block, may be performed.

The display unit 62 is a device that displays the status of the prostheses being processed in the dental prosthesis processing system and the status of the milling devices 40a to 40f used in the dental prosthesis processing system. Conventional display devices such as a liquid crystal display (LCD) panel, organic light emitting diode (OLED) panel, etc., may be used as the display unit 62.

The web server 50 is located at a remote location and is connected to the user terminal 60 and one or more milling devices 40a to 40f through a communication network 70. For example, the web server 50 and the user terminal 60 and/or the milling devices 40a to 40f may be located at remote locations from each other. And the user terminal 60 and each of the milling devices 40a to 40f may be located at remote locations from each other or at the same location. CAM software is mounted on the web server 50. The CAM software of the web server 50 performs a tool path data generation operation to calculate, in other word, to produce the processing path of the processing tool according to status information including (i) device characteristics (for example, tool's initial or reference position, diameter, length, movement trajectory, etc.) and (ii) usage status characteristics (for example, wear degree of the processing tool, current position of the processing tool, etc.) of the processing tool mounted on the milling devices 40a to 40f. The CAM software of the web server 50 controls the milling devices 40a to 40f according to the tool path data to process the workpiece into the shape of the prosthesis. Since the tool path data defines the movement path of the processing tool, it is also referred to as numerical control (NC) data. Therefore, the web server 50 performs a computation operation to obtain the tool path data and a control operation to control the milling devices 40a to 40f. The web server 50 is driven according to user command inputs through the user terminal 60.

As necessary, instead of the user terminal 60, at the web server 50, (1) designing (editing) the shape of a prosthesis in a shape desired by the user, or (2) a nesting operation to place the shape of the prosthesis to be processed on a workpiece, i.e., a block, may be performed.

To control, i.e., drive, the milling devices 40a to 40f using a web server 50 connected to a communication network such as the Internet, a tool path must be calculated, i.e., tool path data must be generated, according to the device characteristics of the milling devices 40a to 40f, more specifically, the device characteristics of the tool mounted on the milling devices 40a to 40f. The device characteristics of the processing tool mounted on the milling devices 40a to 40f are features of the processing tool itself, including one or more of the initial or reference position, diameter, length, and movement trajectory of the processing tool. The device characteristics of the milling devices 40a to 40f considered in calculating the tool path are structural characteristics of the tool mounted on each of the milling devices 40a to 40f and are predetermined characteristics for each milling device 40a to 40f. The device characteristics of the milling devices 40a to 40f include (i) the axis structure of the tool mounted on the milling devices 40a to 40f (for example, whether the tool of the milling devices 40a to 40f moves along orthogonal axes of x, y, z), (ii) the movement range of the tool on each axis (for example, the tool moves in the range of −10.0 mm to +10.0 mm in the x-axis direction), (iii) initial position of each tool (for example, setting the upper center of the processing block as the origin of the tool), (iv) the size (diameter, length, etc.) of the tool mounted on the milling devices 40a to 40f, and the like. The device characteristics of the milling devices 40a to 40f have constant values unless the milling devices 40a to 40f and the tools mounted thereon are replaced, and may be input into the web server 50 as necessary.

In the present invention, in addition to the above-described device characteristics of the milling devices 40a to 40f, the usage status characteristics of the processing tool mounted on the milling devices 40a to 40f are further considered to calculate the tool path. The usage status characteristics of the processing tool are characteristics of the processing tool that occur during the use of the processing tool, such as the degree of wear of the processing tool, the current or changed position of the tool, etc.

The usage status characteristics of the milling devices 40a to 40f include (i) calibration information of the tool mounted on the milling devices 40a to 40f (for example, the measured or corrected position of the tool mounted on the milling devices 40a to 40f), (ii) the degree of wear of the tool mounted on the milling devices 40a to 40f (for example, the number of uses of the tool, usage amount, worn size, etc.), (iii) block information mounted on the milling devices 40a to 40f, and the like.

In this way, when the tool path is calculated, i.e., produced by further considering not only the device characteristics of the milling devices 40a to 40f but also the usage status characteristics of the milling devices 40a to 40f, the tool path can be calculated more precisely and efficiently, thereby improving the dimensional precision of prosthesis processing and reducing the prosthesis processing time.

The milling devices 40a to 40f transmit their own usage status characteristics, along with device characteristics as necessary, to the web server 50 through the communication network 70. The milling devices 40a to 40f also process workpieces (i.e., blocks) such as ceramic blocks into the shape of the prosthesis according to control from the web server 50 through the communication network 70. In the present invention, the number of the milling devices 40a to 40f is not particularly limited as long as they can be controlled by the web server 50, but is preferably six or fewer. Since the milling devices 40a to 40f are typically Chair Side devices installed in spaces where target patients are examined or treated (hospitals, etc.), to fabricate prostheses (restorations) immediately after examination, for example, on the same day, it is necessary to use as many milling devices 40a to 40f as possible, but it is difficult for one web server 50 to simultaneously control more than six milling devices 40a to 40f.

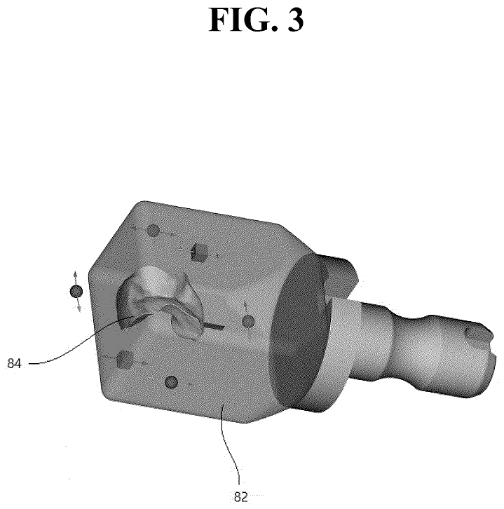

In the present invention, the software of the user terminal 60 or the CAM software of the web server 50 performs a nesting operation according to information (material, size, etc.) of the workpiece, i.e., the block, for processing the prosthesis to generate nesting data in which the shape of the prosthesis to be processed is placed on the block. That is, according to the material, size, etc., of the block, the shape of the required prosthesis is appropriately placed, i.e., nested, inside the block. FIG. 3 is a diagram illustrating a state in which the shape of a prosthesis is placed on a block according to an embodiment of the present invention. As shown in FIG. 3, a nesting operation is performed to place the shape of a desired prosthesis 84 inside the shape of the block 82.

On the other hand, in Chair Side milling devices 40a to 40f used by non-experts in hospitals, etc., it is difficult to replace and mount tools as necessary, so tools of a specific structure (diameter, size, etc.) are already mounted. Therefore, in the present invention, status information of the tools mounted on each of the milling devices 40a to 40f can be transmitted to the web server 50, and the CAM software of the web server 50 calculates the processing path of the processing tool according to the tool status information transmitted from the milling devices 40a to 40f to generate the tool path data.

In this way, when the tool path data corresponding to a specific milling device 40a to 40f is obtained by the CAM software of the web server 50, the web server 50 uses a milling device 40a to 40f in which a block corresponding to the obtained nesting data is mounted and a tool corresponding to the obtained tool path data is mounted, i.e., a milling device 40a to 40f with an appropriate tool, to drive the processing tool of the milling device 40a to 40f according to the nesting data and the tool path data, thereby processing the block 82 to obtain a prosthesis 84 of a desired shape (see FIG. 3).

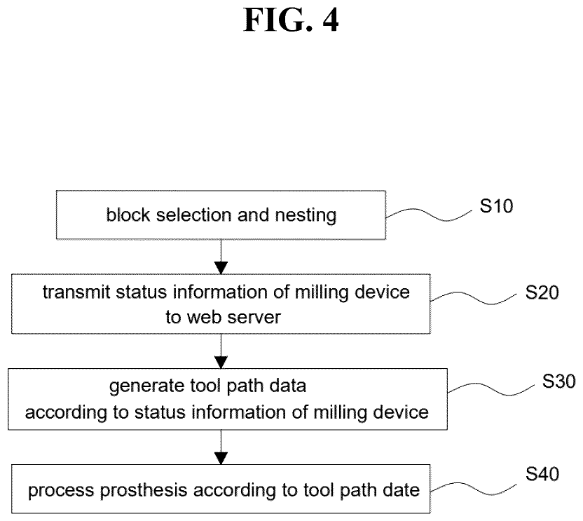

Next, a dental prosthesis processing method according to the present invention will be described with reference to FIG. 4. As shown in FIG. 4, to process a dental prosthesis according to the present invention, first, a nesting step is performed in which a block for processing the prosthesis is selected and the shape of the prosthesis designed in a desired shape is placed on the block (S10). The shape design of the prosthesis can be obtained by conventional CAD (Computer Aided Design) operations by using a patient's oral scan data. The nesting step (S10) may be performed at the user terminal 60 or at the web server 50 connected through the communication network 70. Additionally, the nesting step (S10) may be performed automatically by software of the user terminal 60 or web server 50, or may be performed manually by the user.

Next, status information including the usage status characteristics of one or more milling devices 40a to 40f is transmitted to the web server 50 through the communication network 70 (S20). At this time, device characteristics of one or more milling devices 40a to 40f may be transmitted to the web server 50 together with the usage status characteristics. Alternatively, the device characteristics may be pre-stored in the web server 50. The transmission of the status information may be performed for all milling devices 40a to 40f, or for some available milling devices 40a to 40f, or for a specific milling device 40a to 40f selected by the user.

The transmission of the status information of the milling devices 40a to 40f can be made such that, after the user accesses the web server 50, for example, after the user logs in to the web server 50, the status information of the milling devices 40a to 40f, specifically, the milling device 40a to 40f selected by the user, is transmitted from each milling device 40a to 40f to the web server 50 through the communication network 70.

Next, the web server 50 generates the tool path data (i.e., NC data) to produce the processing path of the processing tool according to status information including the device characteristics (diameter, size, etc.) and the usage status characteristics of the tool mounted on the milling devices 40a to 40f (S30). The tool path data generated in this way is a data particularly suitable for the status of each milling device 40a to 40f. Therefore, the tool path data corresponding to each milling device 40a to 40f should be used for the corresponding milling device 40a to 40f with reference to the unique identification number of each milling device 40a to 40f.

Next, the milling devices 40a to 40f are controlled according to the generated tool path data to process the block into the shape of the prosthesis (S40). The control of the milling devices 40a to 40f may be performed by the user or remotely by the CAM software of the web server 50.

Conventionally, a milling device 40a to 40f supported by the CAM software of the web server 50 is selected, and a tool path is generated according to the status information of the milling device 40a to 40f pre-stored in the CAM software. Therefore, since tool path data is generated according to the general status information of the milling device 40a to 40f, i.e., device characteristics, it fails to reflect changes in the status of the milling device 40a to 40f and only reflects the general device characteristics of the milling device 40a to 40f (for example, reference position of the tool, etc.), resulting in inaccurate tool path data.

In contrast, according to the present invention, since the tool path is calculated by further considering not only the device characteristics of the milling devices 40a to 40f but also the usage status characteristics of the milling devices 40a to 40f, the tool path can be calculated more precisely and efficiently, thereby improving the dimensional precision of prosthesis processing and reducing the prosthesis processing time.

According to the present invention, since a plurality of milling devices 40a to 40f are controlled using the web server 50, when transmitting status information of a plurality of milling devices 40a to 40f to the web server 50, the unique identification number of the device is transmitted together. Thereby, the tool path information corresponding to the transmitted status information can be used only for the device from which the status information was obtained. The status information and/or tool path information of the milling devices 40a to 40f may be transmitted encrypted. Additionally, pre-measured block information may also be transmitted to the web server 50 for remote processing of the milling devices 40a to 40f.

According to the present invention, in operating Chair Side milling devices, for example, milling devices 40a to 40f installed in a hospital, a tool path having an optimized workflow can be generated by the CAM software of the web server 50. The dental prosthesis processing system according to the present invention can more effectively process blocks such as a ceramic block, a lithium disilicate block, a sintered zirconia block, etc.

The present invention has been described above with reference to the accompanying drawings and exemplary embodiments, but the present invention is not limited to the contents shown in the drawings or the above-described embodiments. Although reference numerals are indicated in the claims below to aid understanding, the scope of the claims is not limited to the reference numerals or the contents shown in the drawings, and should be interpreted to cover all modifications, equivalent configurations, and functions of the exemplary embodiments.

What is claimed is:

1. A dental prosthesis processing system comprising:
a user terminal through which commands for performing functions of the dental prosthesis processing system are input;
a web server located at a remote location, connected to the user terminal and one or more milling devices through a communication network, and performing a tool path data generation operation to produce a processing path of a processing tool according to status information including device characteristics and usage status characteristics of the processing tool mounted on milling devices; and
one or more milling devices that transmit their own usage status characteristics to the web server and process a workpiece into the shape of a prosthesis according to the tool path data generated by the web server.

2. The dental prosthesis processing system according to claim 1, further comprising: a display unit that displays the prosthesis and the status of the milling devices.

3. The dental prosthesis processing system according to claim 1, wherein the milling devices are controlled by the web server.

4. The dental prosthesis processing system according to claim 1, wherein the device characteristics of the processing tool are characteristics of the processing tool itself including one or more of an initial or reference position, diameter, length, and movement trajectory of the processing tool, and the usage status characteristics of the processing tool are characteristics of the processing tool that occur during the use of the processing tool mounted on the milling devices.

5. A dental prosthesis processing method comprising the steps of:
nesting step of selecting a block for processing a prosthesis and placing a shape of the prosthesis designed in a desired shape on the block;
transmitting status information including usage status characteristics of one or more milling devices to a web server through a communication network;
generating tool path data for producing a processing path of a processing tool at the web server according to the status information including device characteristics and the usage status characteristics of the processing tool mounted on the milling devices; and
controlling the milling devices according to the generated tool path data to process the block into a shape of the prosthesis.

6. The dental prosthesis processing method according to claim 5, wherein the usage status characteristics of the processing tool mounted on the milling devices are transmitted from each of the milling devices to the web server through the communication network.
